(19) United States
(12) Patent Application Publication
(10) Pub. No.: US 2013/0165838 A1
(43) Pub. Date: Jun. 27, 2013

(54) BLISTER DRESSING INCLUDING A HYDROCOLLOID ADHESIVE BODY

(71) Applicants: Stephane Auguste, Varois et Chaignot (FR); Cecile Richard, Dijon (FR); Sebastien Renard, Dijon (FR); Serge Lecomte, Dijon (FR); Christelle Guillamaud, Chenove (FR)

(72) Inventors: Stephane Auguste, Varois et Chaignot (FR); Cecile Richard, Dijon (FR); Sebastien Renard, Dijon (FR); Serge Lecomte, Dijon (FR); Christelle Guillamaud, Chenove (FR)

(73) Assignee: LABORATOIRES URGO, Chenove (FR)

(21) Appl. No.: 13/643,475

(22) PCT Filed: Apr. 27, 2011
(86) PCT No.: PCT/FR2011/050956

§ 371 Date: Dec. 10, 2012

(30) Foreign Application Priority Data
Apr. 28, 2010 (FR) 1053266

(51) Int. Cl. A61F 13/02

(52) U.S. Cl. CPC A61F 13/0253
USPC 602/54; 424/78.06

(57) ABSTRACT

The present invention relates to a wound dressing which comprises a hydrocolloid adhesive mass comprising a hydrophobic elastomer matrix comprising poly(styrene-olefin-styrene) block copolymers, particles of hydrocolloid(s) dispersed in said elastomer matrix, and at least one tackifying hydrocarbon resin, characterized in that the hydrocolloid adhesive mass has a deformation ratio γ comprised between at least 0.10 and at most 0.40, as measured by subjecting a cylindrical sample having a diameter of 10 mm, of said hydrocolloid adhesive mass having a thickness of 1.0 mm, at a temperature of 37° C. in an atmosphere having a relative humidity of 60%, to a stress of 5000 Pa in order to cause a creep with which is associated a deformation under stress γ_{C} measured after 30 minutes of stress, followed by removing the stress in order to enable recovery characterized by a residual deformation γ_{R} after 30 minutes with no stress, the non-dimensional deformation ratio γ being given by:

$$γ = \frac{{γ}_{C} - {γ}_{R}}{{γ}_{C}}$$

According to one preferential embodiment of the invention, the dressing and the adhesive mass which it contains are in a three-dimensional form having thinned edges.

The subject of the present invention is a novel wound dressing which comprises an adhesive hydrocolloid mass, which dressing is intended for the treatment of wounds such as exudative wounds, burns, superficial or deep dermo-epidermal lesions, which may be chronic or acute, and in particular for the treatment of blisters. The dressings according to the invention exhibit improved staying power over time.

### TECHNICAL BACKGROUND

Wound dressings comprising hydrocolloids have been known for more than 20 years. They consist of a backing on which is deposited an adhesive mass comprising hydrocolloids. By way of example, mention may be made of the products sold under the names Algoplaque® by Laboratoires URGO and Comfeel® by the company Coloplast. Dressings comprising an adhesive mass comprising hydrocolloids, specifically intended for the treatment of blisters, are also known and sold, for example, under the names Urgo Traitement Ampoules® by Laboratoires URGO and Compeed® by the company Johnson & Johnson.

In order to make possible good absorption of the exudates from the wound, these dressings contain relatively high amounts (of the order of 20 to 50% by weight) of hydrocolloids. Preferentially, these dressings are designed to be held in place without the help of an additional adhesive tape, adhering directly to the skin.

The adhesive mass of these known dressings usually consists of a hydrophobic continuous phase, generally based on elastomers, in which is dispersed a noncontinuous phase of hydrocolloid particles intended to absorb the exudates from the wound.

The absorption of the exudates by the hydrocolloids causes the adhesive mass to gel, thereby making it possible to painlessly remove the dressing from the wound after it has been used.

Generally, these adhesive masses comprise, in addition to the elastomer matrix containing hydrocolloid particles, one (or more) compound(s) intended to confer adherence properties on said mass, known as “tackifying” compound(s).

In order to provide maintenance over time of their absorption capacity and of their cohesion during removal, these dressings have a high initial adhesiveness. This is even truer for the dressings intended for the treatment of blisters, which have to be positioned in areas which are curved or difficult to access and which are subject to high mechanical stresses during the use thereof.

Dressings comprising hydrocolloid particles dispersed in an elastomer matrix are, for example, described in documents FR 2 495 473, FR 2 775 903, EP 0 927 051 and EP 1 165 717.

Document EP 0 264 299 describes a dressing having this type of composition in the adhesive mass layer, it being, moreover, specified that the sealing pad, at least around its periphery, is beveled such that its thickness adjacent to its outer edge does not exceed about one quarter of its maximum thickness. WO 92/05755 also describes a dressing comprising hydrocolloid particles dispersed in an elastomer matrix, characterized in that it has a wide peripheral flange with a thickness of less than 0.5 mm and which extends over a distance of at least 10 mm. EP 1 020 198 describes a device similar to that of document WO 92/05755, the peripheral flange portion, having a thickness of 0.15 to 0.20 mm, this time extending up to a distance of 3.0 mm from the thick central portion, having a thickness of 0.5 mm.

### PROBLEM(S) TO BE SOLVED

An ideal dressing must if possible meet a certain number of requirements when it is used by a patient who has a wound, some of which are in essence difficult to meet simultaneously. Too strong an adhesion, although enabling application of the dressing, can result in an undesirable remnant of adhesive mass on the skin when the dressing is removed. Too strong an adhesion can also lead to painful removal of the dressing and/or cause surface desquamation of the skin. Too high a viscosity of the adhesive mass can prevent the latter from following the movements of the body of the wearer, but an adhesive mass that is too fluid will end up going beyond its borders. If a dressing comprising an adhesive mass that is too fluid is subjected to a force perpendicular to its surface, this will cause the mass to creep at the peripheral edges. If such a dressing is subjected to a force in the plane of the dressing, this will cause the dressing to move off-center from its site of application. The backing will slide and may, in certain cases, cause the mass and the backing to delaminate. The intention is in fact to meet as much as possible the needs during the distinct phases of the dressing's life, which can be defined as the contacting, the wearing and the removing.

In the context of the present invention, research has been undertaken in order to develop adhesive masses which give dressings containing them better staying power on the skin. The natural movements of the human body in themselves subject dressings to mechanical forces, said dressings also being subjected to a temperature above ambient temperature and also to the introduction of water and natural greases, without mentioning the exudates from the wounds that they are supposed to isolate from the outside environment. The action of the human body on the dressing often takes place inside clothing or other accessories such as shoes, which increases in particular the mechanical forces. The tendency of the dressings to become detached from the human body naturally increases with the degree of activity of the wearer, sportsmen and sportswomen experiencing a high degree of loss of dressings. While providing good staying power on the skin, it is desirable to have a good initial adhesiveness with a sufficient “tack” to reliably anchor the dressing on the skin when it is initially applied. Preferably, this “tack” will be stable over time, in order to provide the longest possible shelf life after manufacture that can be envisioned. Generally, it is desired to have dressings which exhibit a certain water resistance (thus allowing the wearer to wash at least by means of a shower without losing the dressing), which give the wearer a feeling of protection of the damaged skin and which do not transfer a significant amount of adhesive mass onto the skin. It is also desirable for the adhesive mass to be stable over time, such that, after months, or even years of storage, its properties are not significantly impaired.

### BRIEF DESCRIPTION OF THE FIGURES

FIGS. 1 to 3 represent diagrammatically the respective behaviors of a purely elastic (theoretical) material, of a purely viscous (theoretical) material and of a viscoelastic material.

FIG. 4 represents a thickness profile of a dressing with thinned edges, a preferred embodiment of the present invention.

FIGS. 5 to 9 present results obtained in the creep-recovery test according to the present invention for the Compeed® commercial adhesive mass and also for hydrocolloid adhesive masses according to the formulations of the examples according to the invention and according to the comparative examples of the present application.

FIG. 10 presents results obtained with regard to detachment of oval-shaped blister dressings on volunteer sportsmen wearing them, in a comparative study with the Compeed® commercial product.

FIG. 11 represents a device for measuring the tack of an adhesive material by means of a cylindrical probe.

### SUMMARY OF THE INVENTION

In the context of research aimed at identifying dressings exhibiting good staying power on the skin while at the same time meeting other criteria listed above, the applicant has studied various conditions for modeling adhesive mass behavior. Conditions have been developed which make it possible, surprisingly, to correlate the behavior of a hydrocolloid adhesive mass in creep-recovery with the results, obtained during tests with volunteer wearers, of dressings comprising a layer made up of the adhesive mass in question.

The creep makes it possible to determine how a material deforms over time under a given stress. The recovery characterizes how this material returns to its initial state when it is no longer subjected to the stress.

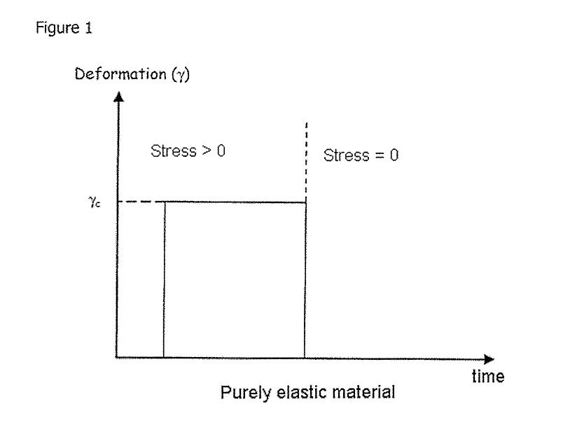

An ideal elastic material responds immediately to a stress and then returns completely to its initial state when it is no longer subjected to a stress (see FIG. 1).

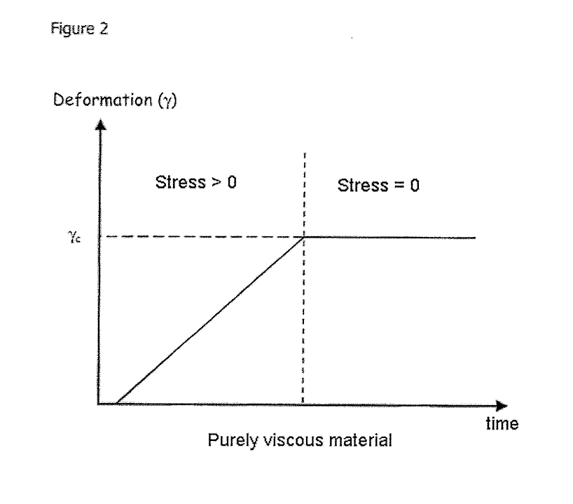

An ideal viscous material deforms gradually over time under the stress, but does not return to its initial state when it is no longer subjected to a stress (see FIG. 2).

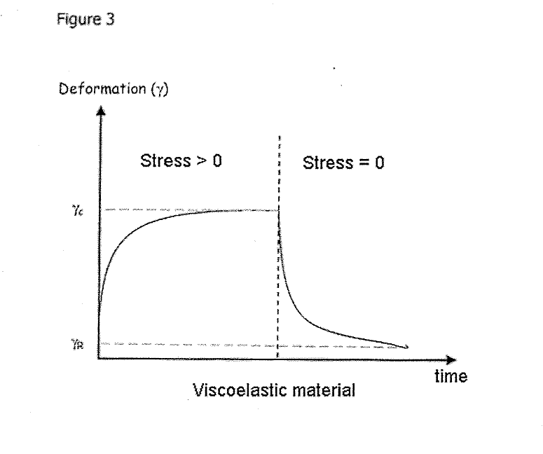

A viscoelastic material has an intermediate behavior (see FIG. 3).

A non-dimensional deformation ratio γ is defined which makes it possible to dispense with the difference in deformation in creep mode γ_{C} and that in recovery mode γ_{R} between the formulations.

$$γ = \frac{{γ}_{C} - {γ}_{R}}{{γ}_{C}}$$

If the material is purely elastic γ_{R}=0 (return to its initial state), then γ=1.

If the material is purely viscous γ_{R}=γ_{C}, then γ≈0.

A viscoelastic material will therefore have a deformation ratio γ comprised between 0 and 1.

The research carried out by the applicant has made it possible to demonstrate that a good correlation between results measured in creep-recovery and the results on voluntary wearers is observed when:
an initial stress of 5000 Pa is applied to the adhesive mass during the measurement of creep-recovery, this stress being unusually high; and
the creep-recovery measurement is carried out at the usual body temperature of 37° C., at normal atmospheric pressure and with a relative humidity of 60%, but without the addition of water to the hydrocolloid adhesive mass. Tests were carried out using a syringe driver capable of continuously depositing the same amount of water on the hydrocolloid adhesive mass during the test as the amount of water absorbed by the same hydrocolloid adhesive mass on a human being wearing it, for the same period of time. Surprisingly, although it might be imagined at the start that a moist medium would more closely mimic the behavior of the skin in vivo, the correlation with the staying power of dressings observed was better for the creep-recovery tests carried out without the addition of water to the adhesive mass.

The present invention therefore relates to a wound dressing which comprises a hydrocolloid adhesive mass comprising the following constituents, the percentages by weight of each constituent being indicated relative to the total weight of the hydrocolloid adhesive mass:
at least 10% and at most 30% of a hydrophobic elastomer matrix comprising poly(styrene-olefin-styrene) block copolymers,
at least 2% and at most 50% of particles of hydrocolloid(s) dispersed in said elastomer matrix,
at least 10% and at most 40% of at least one tackifying hydrocarbon resin,

characterized in that the hydrocolloid adhesive mass has a deformation ratio γ comprised between at least 0.10 and at most 0.40, as measured by subjecting a cylindrical sample having a diameter of 10 mm, of said hydrocolloid adhesive mass having a thickness of 1.0 mm, at a temperature of 37° C. in an atmosphere having a relative humidity of 60%, to a stress of 5000 Pa in order to cause a creep with which is associated a deformation under stress γ_{C} measured after 30 minutes of stress, followed by removing the stress in order to enable recovery characterized by a residual deformation γ_{R} after 30 minutes with no stress, the non-dimensional deformation ratio γ being given by:

$$γ = \frac{{γ}_{C} - {γ}_{R}}{{γ}_{C}}$$

It should be noted here that, although a hydrocolloid adhesive mass thickness of 1.0 mm is used for the standardized measurement of the non-dimensional deformation ratio γ, it is possible to prepare dressings with a variable overall thickness. According to one preferential embodiment, the thickness of the dressing, in its thickest part, comprising the adhesive mass and a protective film and a backing layer, is approximately 0.85 mm.

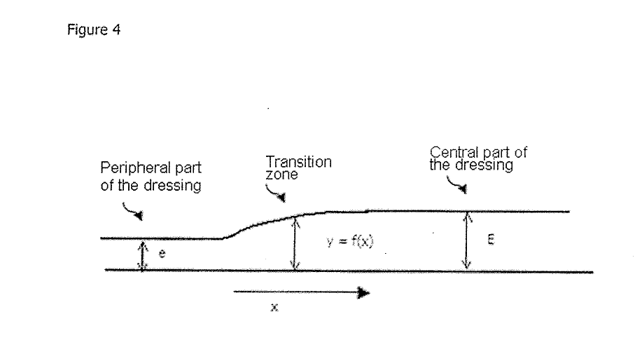

According to another aspect of the present invention, the latter relates to a dressing in which the dressing and the adhesive mass which it contains are presented in a three-dimensional form having thinned edges, such that the height profile of the dressing, going from the geometric center of the dressing to the periphery of the dressing, consists of three sections, as is represented in FIG. 4:
a central part comprising the geometric center of the dressing, in which the height E of the profile (the maximum thickness of the dressing over its central part) is substantially constant;
a peripheral part having a substantially constant thickness e;
a transition zone in which the curvature of connection linking the peripheral and central parts can be represented by a parabolic function of general formula (1):

y=a·x²+b·x+c, with a<0

in which x represents the horizontal distance on the profile (according to the representation given in FIG. 4, x=0 at the beginning of the transition zone), and y represents the height (thickness of the dressing) in the transition zone,

it being specified that:

0.0010 mm⁻¹≦−a≦0.0036 mm⁻¹, and

0.060≦b≦0.130.

A dressing surface could naturally have bumps, but the values to be retained will be those obtained of the parabolic function which has the least possible deviation of the curve observed at the surface (for example by studying under a microscope). Of course, the value “a” in the parabolic function determines the effect of the term in x² and therefore the degree of curvature.

This geometric profile characterized by a parabolic function can in itself confer advantageous technical effects and can be realized independently of the use of a hydrocolloid adhesive mass having a non-dimensional deformation ratio γ comprised between 0.10 and 0.40. However, according to one preferred embodiment of the present invention, the geometric profile is combined with the use of a hydrocolloid adhesive mass having a non-dimensional deformation ratio γ comprised between 0.10 and 0.40, as detailed above.

Without wishing to be bound by a particular theoretical interpretation, the applicant considers that having a profile as indicated above, with neither a flange followed by a very abrupt change in thickness (as described in WO 92/05755 or EP 1 020 198) nor a beveled edge (as in EP 0 264 299), has advantages which make it possible to reconcile resistance to detachment of the dressing with good absorption of exudates. Indeed, poor contacting can, right from the start, cause small pleats in the dressing, in particular at the peripheral edges. These small pleats will allow the entry of air/water/dirt responsible for early detachment. In addition, small pleats make it easier for the dressing to get caught on socks/shoes/straps which cause the dressing to detach. If the dressing does not have small pleats when applied, the rubbing of socks/shoes/straps may be responsible for detachment. According to the curvature, the surface in contact with the sock/shoe/strap will not be the same and the sock/shoe/strap will slide more or less easily over this surface. According to the parabolic profile of this aspect of the present invention, the change from the thickness of the peripheral flange to the maximum thickness takes place gradually. There is no abrupt change and this allows better sliding. A gradual change with a beveled edge (as described in patent EP 0 264 299) requires a very shallow slope, which creates a smaller thickness of the mass over a greater surface area and, consequently, a reduced exudate absorption (because it is the particles of hydrocolloid(s) contained in the mass which enable good exudate absorption).

The dressing seen from above can have a square or rectangular shape. Likewise, its size can be freely adjusted according to the surface area of the part to be treated or to be protected. For example, a dressing intended for the treatment of blisters can have a rectangular shape approximately 7 cm long and approximately 4 cm wide, while a dressing intended for the treatment of ulcers can suitably have a 10 cm×10 cm square shape. Shapes other than square or rectangular are also envisioned for dressings according to the invention, for example circular or oval shapes, or shapes having the appearance of a bean.

In the case of a rectangular or oval shape, two distinct axes passing through the geometric center are clearly distinguished, firstly a transverse axis, of which the length from one peripheral edge to the other edge, passing through the center, is as short as possible (transverse axis), and secondly the longitudinal axis, perpendicular to the transverse axis and longer than the latter.

According to one preferential embodiment of the present invention, the dressing, in particular of oval or rectangular type, comprises distinct longitudinal and transverse axes passing through the geometric center of the dressing, in which:
on the longitudinal axis, 0.0010 mm⁻¹≦−a≦0.0016 mm⁻¹, and 0.060≦b≦0.0920, and
on the transverse axis, 0.0020 mm⁻¹≦−a≦0.0036 mm⁻¹, and 0.080≦b≦0.130.

In this preferential embodiment, the curvature of connection in the longitudinal direction is therefore smaller than in the transverse direction.

Preferably, in the dressings having thinned edges in this embodiment of the present invention, the height E in the central part of the dressing of substantially constant thickness is at least 0.80 mm and at most 1.2 mm, and the height e in the peripheral part of the dressing of substantially constant thickness is at least 0.26 mm and at most 0.40 mm.

Dressings having geometric profiles as defined by the parabolic profile above can in particular be obtained by implementing the process of patent application WO 2010/004222 by the applicant, a cylinder which comprises the imprint of the desired geometric shapes being used to prepare such dressings having thinned edges.

### DETAILED DESCRIPTION OF THE INVENTION

The dressings according to the present invention comprise a layer of hydrocolloid adhesive mass comprising the essential elements represented by the hydrophobic elastomer matrix, the particles of hydrocolloid(s) and also at least one tackifying hydrocarbon resin as indicated above. The dressings according to the present invention can also have a backing on the face of the hydrocolloid adhesive mass not intended to be in contact with the patient's skin. The dressings according to the present invention can also have a protective film on the face of the hydrocolloid adhesive mass intended to be in contact with the patient's skin. According to one embodiment of the present invention, the dressing has a shape which has thinned edges. The dressing seen from above can have a square or rectangular shape. Likewise, its size can be freely adjusted according to the surface area of the part to be treated or to be protected. For example, a dressing intended for the treatment of blisters can have a rectangular shape approximately 7 cm long and approximately 4 cm wide, while a dressing intended for the treatment of ulcers can suitably have a 10 cm×10 cm square shape. Shapes other than square or rectangular are also envisioned for dressings according to the invention, for example circular or oval shapes or shapes which have the appearance of a bean.

The elastomer matrix of the hydrocolloid adhesive mass layer comprises poly(styrene-olefin-styrene) block copolymers. Preferably, the elastomer matrix comprises one (or more) elastomer(s) belonging to the family of poly(styrene-isoprene-styrene) triblock copolymers (abbreviated to: poly(SIS)) and blends of poly(SIS) triblock copolymers and of poly(styrene-isoprene) diblock copolymers, and in particular poly(SIS)s having a styrene content comprised between 14 and 52% and preferably between 14 and 30% by weight relative to the weight of said poly(SIS).

Such products well known to those skilled in the art are, for example, sold by the company Kraton under the name Kraton® D or by the company Dexco Polymers LP under the name Vector®.

Among the preferred poly(SIS) triblock copolymers, mention may in particular be made of the products sold under the names Kraton® D-1111K, Kraton® D-1111CS, Kraton® D-1107 or Kraton® 1161, Vector® 4114 and Vector® 4113.

Poly(styrene-butadiene-styrene) triblock copolymers can also be used in the context of the invention.

Among these poly(styrene-butadiene-styrene) copolymers, mention may in particular be made of the product sold under the name Kraton® D-1102 by the company Kraton.

Preferably, the elastomers forming the elastomer matrix will be present, within the adhesive mass of the dressings according to the invention, in an amount of from 10 to 30% by weight, and preferably from 15 to 25% by weight, of the total weight of the hydrocolloid adhesive mass.

Generally, the abovementioned elastomer matrix incorporates one (or more) hydrocolloid(s).

The term “hydrocolloid” is intended to denote herein any compound normally used by those skilled in the art for its ability to absorb hydrophilic liquids such as water, physiological saline or exudates from a wound.

By way of example of a hydrocolloid that can be used in the context of the invention, mention may be made of pectin, alginates, natural plant gums such as, in particular, karaya gum, cellulose derivatives such as, in particular, carboxymethylcelluloses and their alkali metal, in particular sodium or calcium, salts, and also synthetic polymers based on acrylic acid salts, known as “superabsorbents”, such as, in particular, the products sold by the company BASF under the name Luquasorb® 1003 or by the company Ciba Specialty Chemicals under the name Salcare® SC91. Of course, mixtures of these products can be used as hydrocolloids.

The preferred hydrocolloids in the context of the present invention are the alkali metal salts of carboxymethylcellulose, and in particular sodium carboxymethylcellulose.

The amount of hydrocolloid(s) incorporated into the elastomer matrix will be adjusted according to the desired level of absorption. Generally, the amount of hydrocolloid(s) may be of the order of from 2 to 50% by weight, relative to the total weight of the hydrocolloid adhesive mass.

In the context of the present invention, an amount of hydrocolloid(s) comprised between 20 and 50% by weight relative to the total weight of the hydrocolloid adhesive mass will preferably be used.

The elastomer matrix containing hydrocolloid particles is made adhesive through the addition of “tackifying” products. Tackifying products which can be used in the context of the present invention are tackifying resins of hydrogenated hydrocarbon(s). Such products are sold, for example, by the company Arakawa under the name Arkon®. These products are sold in grades indicating the softening temperature. The softening point temperature of Arkon® P 90 is 90° C., that of Arkon® P 125 is 125° C. and that of Arkon® P 140 is 140° C. The Arkon® P 100 and Arkon® M 100 resins are prepared by hydrogenation of aromatic hydrocarbon resins obtained by cationic polymerization of the C9 fraction with boiling points comprised between 140 and 280° C., this fraction being substantially free of C5 fraction. In the case of the Arkon® P 100 resin, the degree of hydrogenation is considered to be total, all the aromatic rings being hydrogenated. In the case of the Arkon® M 100 resin, the hydrogenation is incomplete, the degree of hydrogenation being from 50 to 80%. Since the starting products before hydrogenation of tackifying resins such as those of the Arkon® series are aromatic, such resins are also commonly called “hydrogenated aromatic tackifying resins”, although the final product may no longer contain an aromatic ring.

In one preferential embodiment of the present invention, at least two tackifying resins of hydrogenated hydrocarbon(s) must be present, the softening points of which differ by at least 10° C. and by at most 40° C., preferably by at least 20° C. and by at most 40° C., even more preferentially by at least 30° C. and by at most 40° C. They are preferably two tackifying resins in the class of hydrogenated aromatic resins as detailed above. Preferentially, the softening point of the tackifying resin of which the softening point is the lowest will be at least 65° C., preferably at least 80° C. Preferentially, the softening point of the tackifying resin of which the softening point is the highest will be at most 145° C., preferably at most 130° C.

It has been observed that the use of a single hydrogenated aromatic tackifying resin can reduce the ability to exhibit a good initial adhesiveness with a sufficient “tack” to reliably anchor the dressing on the skin at the time it is initially applied, and also to retain this initial adhesiveness at the time of application, over a long dressing shelf life. The use of two hydrogenated aromatic tackifying resins makes it possible to reconcile as successfully as possible the set of desired properties.

In addition to the tackifying resins of hydrogenated (aromatic) hydrocarbon(s), the hydrocolloid adhesive mass can optionally contain other families of tackifying resins, for example the products obtained by polymerization of C5 aliphatic monomers (such as the Wingtack® series), polyterpenes or rosin resins. Among the tackifying resins used in the adhesives field, a distinction is generally made between, on the one hand, (synthetic) hydrocarbon resins and, on the other hand, resins of natural origin (although the latter can be chemically modified). Among the (synthetic) hydrocarbon resins are in particular:
resins of aromatic/C9 type (previously described in detail and applicable in dressings according to the invention). These resins are generally obtained by hydrogenation of resins obtained by polymerization of aromatic monomers, such as styrene, α-methylstyrene, vinyltoluene, indene and methylindene;
resins of aliphatic/C5 type, generally obtained by polymerization of monomers such as isoamylene, cyclopentene and piperylene;
resins based on indene and on coumarone; and
DCPD resins based on dimers of cyclopentadiene.

Among the resins of natural origin are in particular:
resins of (poly)terpene type, obtained by polymerization of terpene monomers (such as pinene, camphene or limonene), optionally with a synthetic comonomer, such as styrene, and/or modified by reaction with a phenol; and
rosin resins. The term “rosin” generally refers to a substance obtained from resinous trees such as pine trees. The resin obtained is predominantly composed of organic acids of the diterpene family. Various diterpene compounds may be present, the major backbone being that of abietic acid. It is possible to hydrogenate resinic acids such as abietic acid and its congeners, which in the natural state have C═C bonds. In addition, the —CO₂H group in the resinic acids can be esterified, and the esters with polyols (glycols, pentaerythritol, etc.) are well known.

Preferably, in the dressings of the present invention, resins other than tackifying resins of hydrogenated (aromatic) hydrocarbon(s) constitute only at most 5% by weight relative to the total weight of the hydrocolloid adhesive mass, and more preferentially the hydrocolloid adhesive mass is substantially free of tackifying resins other than tackifying resins of hydrogenated hydrocarbon(s).

Preferably, the tackifying product(s) will represent from 10 to 40% by weight, of the total weight of the hydrocolloid adhesive mass, preferentially from 25 to 35%. Preferably, in the preferential embodiment according to which there are two tackifying resins of hydrogenated hydrocarbon(s), each of the two tackifying resins of hydrogenated hydrocarbon(s), the softening points of which differ by at least 10° C. and by at most 40° C., will represent at least 5%, preferably at least 10% by weight of the total weight of the hydrocolloid adhesive mass.

Various additional compounds may be added to the elastomer matrix containing the abovementioned tackifying compounds and hydrocolloids in order to obtain hydrocolloid adhesive masses which have optimized elasticity, adhesion, stability over time and cohesion properties.

Such compounds are, for example, stabilizers such as, in particular, antioxidants, and plasticizers such as, in particular, plasticizing oils.

The term “stabilizer” is intended to denote herein any compound capable of providing the stability, with respect to oxygen (antioxidant), heat, ozone and ultraviolet radiation, of the compounds used in the formulation of the hydrocolloid adhesive masses, in particular of the tackifying resins and of the block copolymers. These stabilizing compounds are well known and may be used alone or as a mixture.

Among the antioxidant compounds which can be used according to the invention, mention may be made of phenolic antioxidants, such as, for example, the products sold by the company Ciba-Geigy under the names Irganox® 1010, Irganox®565 and Irganox® 1076, and also sulfur-comprising antioxidants, such as, for example, zinc dibutyldithiocarbamate, sold by the company Flexsys under the name Perkacit® ZDBC.

These compounds can be used alone or as a mixture, preferably in a proportion of from 0 to 2% by weight, and more particularly from 0.1 to 0.6% by weight, relative to the total weight of the hydrocolloid adhesive mass.

In the context of the present invention, use will preferably be made of the combination of Irganox®1010 and Perkacit®ZDBC.

Among the plasticizing compounds which can be used according to the invention, mention may be made of the plasticizers normally used by those skilled in the art in the preparation of hydrocolloid adhesive masses, plasticizing oils or else phthalate derivatives, such as dioctyl phthalate, or else adipates.

The use of plasticizing oils is particularly preferred in the context of the present invention.

The term “plasticizing oil” is intended to denote herein the mineral or vegetable oils commonly used by those skilled in the art to plasticize the block copolymers of the styrene-olefin-styrene type used in the composition of hydrocolloid adhesive masses.

These mineral oils generally consist of mixtures in variable proportions of compounds of paraffinic, naphthenic or aromatic nature.

Among the plasticizing oils which can be used according to the invention, mention may be made of the products sold by Shell under the names Ondina® and Risella®, which consist of mixtures based on naphthenic and paraffinic compounds, or under the name Catenex®, which consist of mixtures based on naphthenic, aromatic and paraffinic compounds, the products sold by Croda under the name Crodamol DOA or sold by Eigenmann & Veronelli under the trade name Lincol DOA-C, which are diethylhexyl adipate.

In the context of the present invention, use will preferentially be made of the diethylhexyl adipate sold under the name Crodamol DOA by Croda.

These plasticizing compounds can be used alone or as a mixture, preferably in a proportion of from 5 to 20% by weight, and more particularly from 7 to 15% by weight, relative to the total weight of the hydrocolloid adhesive mass.

The hydrocolloid adhesive mass of the dressings according to the invention can also comprise one or more surfactant compound(s) in an amount of less than or equal to 10% by weight, preferably less than or equal to 5% by weight, relative to the total weight of the hydrocolloid adhesive mass.

A preferred surfactant compound in the context of the present invention is the compound sold under the name AcResin®.

Another preferred surfactant compound in the context of the present invention is polysorbate 80, such as, for example, the product sold by the company SEPPIC under the name Montanox® 80.

The thickness of the layer of hydrocolloid adhesive mass of the dressings according to the invention is variable, depending on the use envisioned. Preferably, the thickness of the layer of hydrocolloid adhesive mass will vary between at least 0.3 mm and at most 2.0 mm. The high thicknesses, of 1.2 mm and above, for example between 1.2 mm and 1.5 mm, will be preferred for chronic wounds and in other situations in which the volume of the exudates expected is high. Although the thickness of the layer of hydrocolloid adhesive mass can be substantially the same over the entire surface area of the dressing according to one embodiment of the present invention, according to another embodiment the thickness is reduced at the edges.

The dressings of the invention may be of different types, but will preferably comprise a backing.

Generally, the backing will be chosen according to the required properties (leaktightness, elasticity, etc.) in the desired application.

Thus, the dressing according to the invention can comprise a backing, such as a film formed of one or more layers and with a thickness which can vary from 5 to 150 μm, a nonwoven or else a foam having a thickness of from 10 to 500 μm, onto which the hydrocolloid adhesive mass has been coated in a continuous or noncontinuous fashion. Most commonly, the backing will have an overall thickness of from 10 to 100 μm, preferably from 20 to 50 μm.

These backings based on synthetic or natural materials are well known to those skilled in the art.

Among the backings in the form of a foam which can be used in the context of the invention, mention may thus be made of polyethylene, polyurethane or PVC foams, for example.

Among the nonwoven backings which can be used in the context of the invention, mention may be made of nonwovens made of polypropylene, polyethylene, polyurethane, polyamide or polyester, for example.

In the context of the present invention, use will preferably be made of backings in the form of films, and in particular polyurethane films, such as, for example, the films sold by the company Smith and Nephew under the reference Lasso® which are produced from polyurethane sold by the company BF Goodrich under the name Estane®; low-density polyethylene films, such as, for example, the films sold by the company SOPAL; films based on thermoplastic polyether/polyester copolymer, such as, for example, the products sold by the company Dupont de Nemours under the name Hytrel®; or else complex films combining a polyurethane film and a nonwoven.

According to one embodiment variant of the invention, the dressing can comprise an absorbent layer positioned between the backing and the hydrocolloid adhesive mass. This absorbent layer can consist of any type of absorbent material, such as, for example, a foam (such as, in particular, a polyurethane foam), a nonwoven, a superabsorbent polymer layer, or a combination of these materials. An absorbent layer of this type, which can make it possible to absorb large volumes of exudates, can have a thickness ranging from 50 μm up to 2.5 mm.

The dressings according to the invention can comprise a protective film on the face of the layer of hydrocolloid adhesive mass intended to be in contact with the patient's skin. Said film can advantageously consist of a silicone film or of a silicone-treated paper. The silicone films are often prepared from polyesters. The papers for silicone-treated paper are generally herein dense supercalendered papers. Complex films based on both paper and plastic film also exist.

The protective film of a dressing according to the invention will preferably have a thickness comprised between at least 30 μm and at most 300 μm, so that it can be grasped.

The dressings according to the present invention are preferably manufactured according to the following process:

a) a first laminated element is formed by securing together a backing film and the extruded hydrocolloid adhesive mass;

b) said laminated element is shaped to obtain an intermediate product comprising a plurality of zones, each forming an incipient dressing;

c) a second laminated element is formed by securing together said intermediate product and a protective film;

d) said second laminated element is cut so as to thereby form a plurality of dressings.

### EXEMPLARY EMBODIMENTS

### Comparative Example of Formulation 1

A hydrocolloid adhesive mass consisting of the following compounds was prepared (amount expressed by weight per 100 grams of mass):

| | | Amount (by |
|---|---|---|
| | | weight per |
| No. | Compounds | 100 grams) |
| 1 | Elastomer | 17.8 |
| | Styrene-isoprene-styrene/styrene-isoprene block | |
| | copolymer (sold under the name Vector ® 4114A | |
| | by Dexco Polymers LP) | |
| 2 | Plasticizer | 8.6 |
| | Mineral oil (sold under the name Edelex ® 945 by | |
| | Shell) | |
| 3 | Surfactant | 5.0 |
| | UV-crosslinkable acrylic ester copolymer (sold | |
| | under the name AcResin ® A258UV by BASF) | |
| 4 | Tackifying resin | 35.0 |
| | Synthetic hydrocarbon resin (sold under the name | |
| | Wingtack ® 86 by Safic Alcan) | |
| 5 | Tackifying resin | 5.0 |
| | Synthetic hydrocarbon resin (sold under the name | |
| | Wingtack ® 10 by Safic Alcan) | |
| 6 | Hydrocolloid | 28.0 |
| | Carboxymethylcellulose (sold under the name | |
| | CMC Blanose ® 7H4XF by Hercules) | |
| 7 | Antioxidant | 0.3 |
| | Zinc dibutyldithiocarbamate (sold under the name | |
| | Perkacit ® ZDBC by the company Flexsys) | |
| 8 | Antioxidant | 0.3 |
| | Pentaerythritol tetrakis (3-(3,5-di(tert-butyl)-4- | |
| | hydroxyphenyl)propionate) (sold under the name | |
| | Irganox ® 1010 by Ciba Speciality Chemicals) | |

This hydrocolloid adhesive mass was prepared by carrying out the following process:

Compounds 1, 2, 7 and 8 were introduced into a Z-arm blender at a set temperature of 130° C.

Compound 3 was added at the 40^{th} minute.

Compound 4 was added at the 50^{th} minute.

Compound 5 was added at the 60^{th} minute.

Compound 6 was added at the 70^{th} minute.

The blender was emptied at the 80^{th} minute.

The hydrocolloid adhesive mass was heated to a temperature of the order of 110 to 130° C.

### Comparative Example of Formulation 2

A hydrocolloid adhesive mass consisting of the following compounds was prepared (amount expressed by weight per 100 grams of mass):

| | | Amount (by |
|---|---|---|
| | | weight per |
| No. | Compounds | 100 grams) |
| 1 | Elastomer | 18.0 |
| | Styrene-isoprene-styrene/styrene-isoprene block | |
| | copolymer (sold under the name Kraton ® D 1111 | |
| | K by Kraton) | |
| 2 | Plasticizer | 7.0 |
| | Diethylhexyl adipate (sold under the name | |
| | Crodamol DOA by Croda) | |
| 3 | Tackifying resin | 13.0 |
| | Resin (sold under the name Arkon P 140 ® by | |
| | Arakawa) | |
| 4 | Tackifying resin | 20.0 |
| | Resin (sold under the name Arkon P 90 ® by | |
| | Arakawa) | |
| 5 | Hydrocolloid | 41.0 |
| | Carboxymethylcellulose (sold under the name CMC | |
| | Blanose ® 7H4XF by Hercules) | |
| 6 | Antioxidant | 0.5 |
| | Zinc dibutyldithiocarbamate (sold under the name | |
| | Perkacit ® ZDBC by the company Flexsys) | |
| 7 | Antioxidant | 0.5 |
| | Pentaerythritol tetrakis (3-(3,5-di(tert-butyl)-4- | |
| | hydroxyphenyl)propionate) (sold under the name | |
| | Irganox ® 1010 by Ciba Speciality Chemicals) | |

This hydrocolloid adhesive mass was prepared by carrying out the following process:

Compounds 1, 2, 6 and 7 were introduced into a Z-arm blender at a set temperature of 135° C.

Compounds 3 and 4 were added at the 45^{th} minute.

Compound 5 was added at the 55^{th} minute.

The blender was emptied at the 65^{th} minute.

### Example 1 of Formulation According to the Invention

A hydrocolloid adhesive mass consisting of the following compounds was prepared (amount expressed by weight per 100 grams of mass):

| | | Amount (by |
|---|---|---|
| | | weight per |
| No. | Compounds | 100 grams) |
| 1 | Elastomer | 18.0 |
| | Styrene-isoprene-styrene/styrene-isoprene block | |
| | copolymer (sold under the name Kraton ® D 1111 | |
| | K by Kraton) | |
| 2 | Plasticizer | 10.0 |
| | Diethylhexyl adipate (sold under the name | |
| | Crodamol DOA by Croda) | |
| 3 | Tackifying resin | 15.0 |
| | Resin (sold under the name Arkon P 125 ® by | |
| | Arakawa) | |
| 4 | Tackifying resin | 15.0 |
| | Resin (sold under the name Arkon P 90 ® by | |
| | Arakawa) | |
| 5 | Hydrocolloid | 41.0 |
| | Carboxymethylcellulose (sold under the name CMC | |
| | Blanose ® 7H4XF by Hercules) | |
| 6 | Antioxidant | 0.5 |
| | Zinc dibutyldithiocarbamate (sold under the name | |
| | Perkacit ® ZDBC by the company Flexsys) | |
| 7 | Antioxidant | 0.5 |
| | Pentaerythritol tetrakis (3-(3,5-di(tert-butyl)-4- | |
| | hydroxyphenyl)propionate) (sold under the name | |
| | Irganox ® 1010 by Ciba Speciality Chemicals) | |

This hydrocolloid adhesive mass was prepared by carrying out the following process:

Compounds 1, 2, 6 and 7 were introduced into a Z-arm blender at a set temperature of 135° C.

Compounds 3 and 4 were added at the 45^{th} minute.

Compound 5 was added at the 55^{th} minute.

The blender was emptied at the 65^{th} minute.

### Example 2 of Formulation According to the Invention

A hydrocolloid adhesive mass consisting of the following compounds was prepared (amount expressed by weight per 100 grams of mass):

| | | Amount (by |
|---|---|---|
| | | weight per |
| No. | Compounds | 100 grams) |
| 1 | Elastomer | 18.0 |
| | Styrene-isoprene-styrene/styrene-isoprene block | |
| | copolymer (sold under the name Kraton ® D 1111 | |
| | K by Kraton) | |
| 2 | Plasticizer | 10.0 |
| | Diethylhexyl adipate (sold under the name | |
| | Crodamol DOA by Croda) | |
| 3 | Tackifying resin | 30.0 |
| | Resin (sold under the name Arkon P 125 ® by | |
| | Arakawa) | |
| 4 | Hydrocolloid | 41.0 |
| | Carboxymethylcellulose (sold under the name CMC | |
| | Blanose ® 7H4XF by Hercules) | |
| 5 | Antioxidant | 0.5 |
| | Zinc dibutyldithiocarbamate (sold under the name | |
| | Perkacit ® ZDBC by the company Flexsys) | |
| 6 | Antioxidant | 0.5 |
| | Pentaerythritol tetrakis (3-(3,5-di(tert-butyl)-4- | |
| | hydroxyphenyl)propionate) (sold under the name | |
| | Irganox ® 1010 by Ciba Speciality Chemicals) | |

This hydrocolloid adhesive mass was prepared by carrying out the following process:

Compounds 1, 2, 5 and 6 were introduced into a Z-arm blender at a set temperature of 135° C.

Compound 3 was added at the 45^{th} minute.

Compound 4 was added at the 55^{th} minute.

The blender was emptied at the 65^{th} minute.

Although this formulation meets the creep-recovery measurement criteria (value of γ comprised between 0.10 and 0.40 according to the measurement method of the present invention) and exhibits good staying power on the skin, it appears to be less capable of exhibiting a good initial adhesiveness with a sufficient “tack” to reliably anchor the dressing on the skin at the time it is initially applied, and of retaining this initial adhesiveness at the time of application, over a long dressing shelf life. An adhesive mass comprising two tackifying resins of hydrogenated hydrocarbon(s), the softening points of which differ by at least 10° C. and by at most 40° C., an illustrative example of which is given in the example of formulation 1 according to the invention above, therefore currently constitutes a particularly preferred embodiment.

### Evaluation of the Properties of Adhesive Masses According to the Formulation Examples

Procedure

A 1 mm-thick sheet of mass for each formulation is prepared using a Collin 200E heated press (type of heating press) at 110° C.

Creep

Sample cut out with a hole punch 20 mm in diameter.

Rheometer: Gemini 150

Spindle: Plate 20 mm

Temperature: 37° C.

Stress: 5000 Pa

Creep time: 30 min

Recovery time: 30 min

The creep of the product sold by Johnson&Johnson under the trade name Compeed® was also measured.

According to the present invention, this creep-recovery test is carried out in a dry medium.

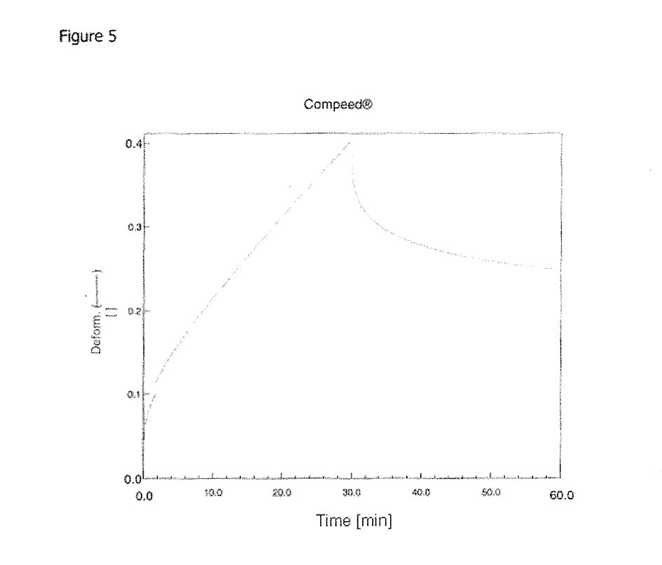

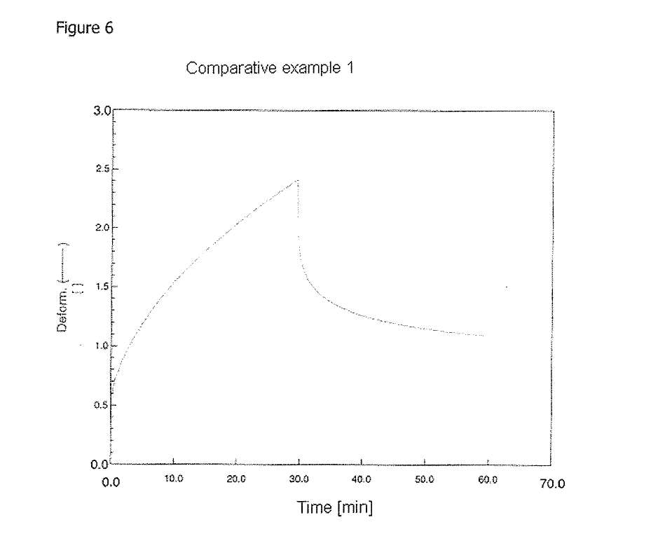

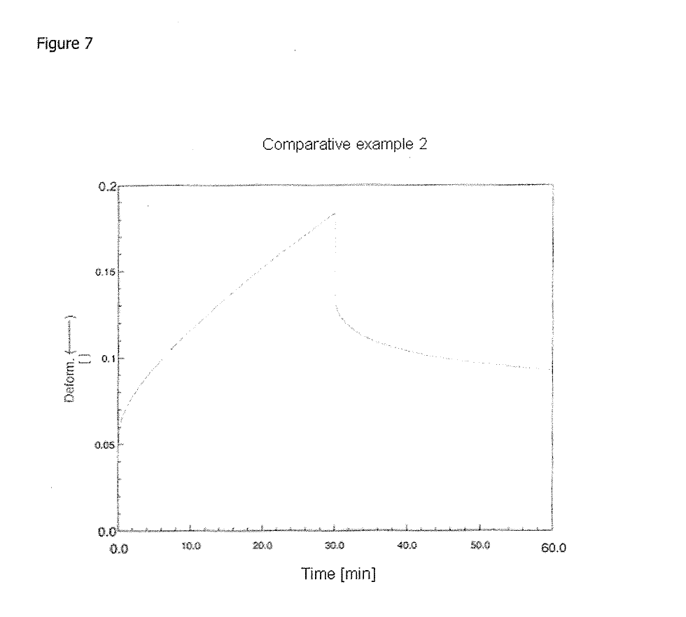

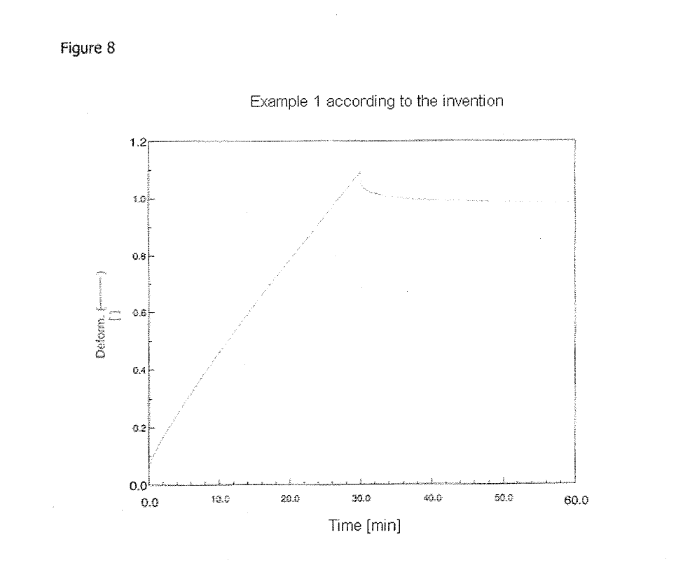

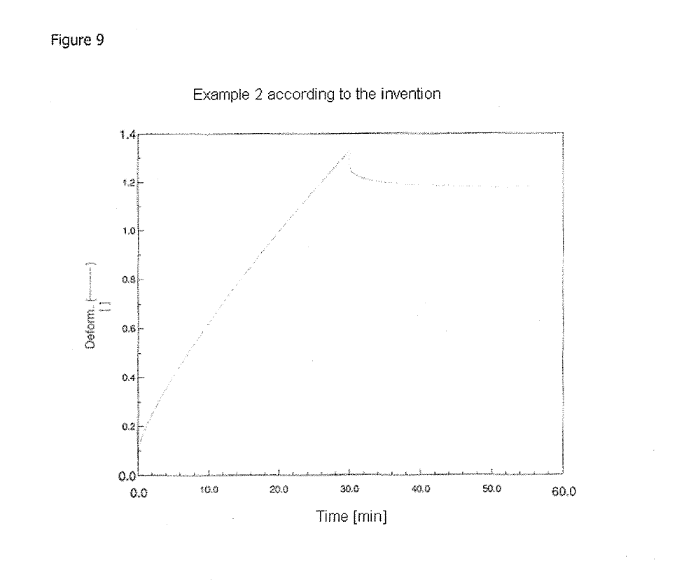

The results obtained are given in FIGS. 5 to 9 and summarized in the following table:

| Formula | γ_{C} | γ_{R} | γ |
|---|---|---|---|
| Compeed ® | 0.400 | 0.222 | 0.44 |
| Comparative example 1 | 2.553 | 1.231 | 0.52 |
| Comparative example 2 | 0.183 | 0.093 | 0.50 |
| Example 1 according to the invention | 1.100 | 0.720 | 0.35 |
| Example 2 according to the invention | 1.300 | 1.150 | 0.12 |

It was found that the formulae of comparative examples 1 and 2 exhibit less resistance to use compared with the Compeed® product (in the type of test on wearers that will be described hereinafter) and show a higher deformation ratio. These formulae have a more elastic behavior in creep/recovery mode.

The formulae of examples 1 and 2 according to the invention, which appear to have a better staying power than Compeed® in tests on wearers, exhibit a lower deformation ratio. These formulae appear to have a more viscous behavior in creep/recovery mode.

### Results on Wearers

Dressings comprising the hydrocolloid adhesive mass according to example 1 were tested by volunteer wearers performing sporting activities. It emerged therefrom that the dressings according to the invention exhibit a reduced degree of detachment and a certain resistance to water, confer a feeling of protection of the skin (protection against pressures, rubbing by shoes) and do not transfer a significant amount of adhesive mass onto the skin.

In one study, protective fingerlifts of an adhesive mass of an oval-shaped blister dressing of the Compeed® brand were removed and replaced with neutral fingerlifts. A blister dressing also oval shaped and of the same size and thickness was prepared with the adhesive mass according to the example of formulation 1 of the present invention. Thirty-two individuals participated in this first study which began with running for 1 h or 1 h 30 and then continued over 48 h under urban conditions with, however, a further sporting activity (walking or running) for 50% of the individuals.

Immediately after the running, a significant difference is observed between the two dressings in terms of the state of detachment: the dressings according to the invention detach less than the Compeed® dressings and this is confirmed after they have been worn for 48 h. Indeed, 63% of the dressings according to the invention are still in place after having been worn for 48 h and showed little detachment, whereas 47% of the Compeed® dressings are present with a variable state of detachment.

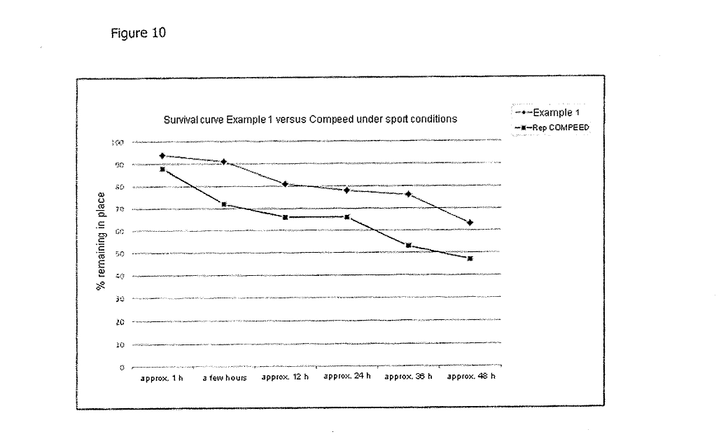

The results of this first study are presented in FIG. 10.

Other similar tests with volunteer sportsmen wearing the dressings, comparing bean and oval shapes with the same hydrocolloid adhesive mass according to the example of formulation according to the invention, made it possible to demonstrate that the shape has no impact on the staying power of the dressing (profile of loss of dressing over time).

### Study of the Aging of the Dressings

The adhesive strength of the dressings as prepared in the example of formulation 1 and the example of formulation 2 according to the invention was measured in order to evaluate the difference in behavior after aging for 18 months and 24 months in an incubator at 21±2° C. at a relative humidity of 60±15% RH.

To this end, use was made of the “probe tack” method, which is aimed at measuring the detachment of a cylindrical probe applied to an adhesive with a pressure stress P₀ for a given time t under predetermined temperature and humidity conditions.

Equipment

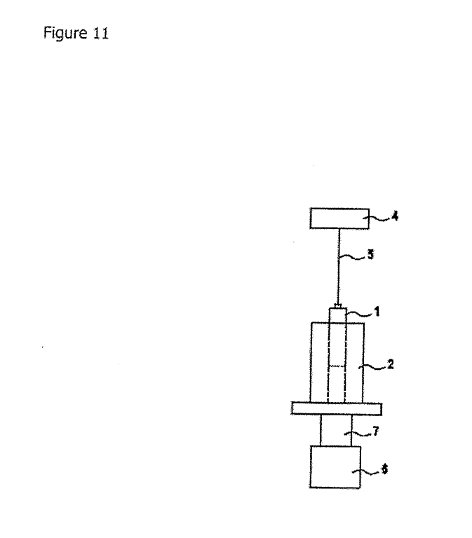

More specifically, this method was carried out using the device represented in FIG. 11.

This device essentially consists:
of a cylindrical probe 1 exhibiting a finely polished end capable of moving longitudinally (from the bottom upwards in the example represented) through a probe guide 2 consisting of a cylinder pierced in its center with a free hole which allows the free frictionless passage of the probe;
of an electronic dynamometer comprising a movable gripping element 4 which can be connected to the probe 1 via a small chain 5 and a base 6 on which a plate 7 can be fitted, said dynamometer being in addition connected to a system for acquiring and recording data, such as, in particular, the rate of descent of the probe, the contacting pressure of the probe on the adhesive, the contact time between the probe and the adhesive and the rate of rise of the probe.

Samples

Mass coated on a film of Exopack inspire 2304 polyurethane PU film 30 μm thick.

The samples cut out have a surface area greater than the base of the probe guide.

Procedure:

The plate 7 was fitted to the base 6 of the dynamometer.

The small chain 5 of the probe was attached to the movable gripping element 4 of the dynamometer.

The probe 1 was cleaned with a solvent (this operation being repeated before each measurement).

The sample was applied to the base of the probe guide with the test face facing said base.

The parameters of the dynamometer (rate of descent, contacting pressure, contact time and rate of rise) were programmed.

The measurement was carried out by performing the following operations: descent of the probe.

The detachment force was recorded in kPa and was translated into tear-off stress S according to the relationship:

S=F/A

where F=Force expressed in N and A=Area expressed in m².

Parameters of the Test:

Application stress: 4.3 kPa (brass probe with a weight of 35 g and a diameter of 10 mm)
Test rate: 300 mm/min
Contact time: 5 s
The measurements were carried out on 10 samples of each of the masses.

Results:

The means of the results of the measurements thus carried out, expressed in kPa, have been given in table I.

The aging behavior was evaluated comparatively between the mass of the example of formulation 1 and the example of formulation 2 according to the invention on two protector configurations. Silphan is a silicone-coated polyester PET protective film and R1010 is a silicone-treated supercalendered paper.

The purpose of the analysis carried out with two protectors is to show that one of the two masses actually exhibits a significantly weaker aging behavior than the other, independently of the surface finish generated by the protectors.

The data resulting from the probe tack measurements show a very different aging behavior between the mass of the example of formulation 2 and the example of formulation 1 according to the invention. The table shows that the level of tack is constant on the mass of example 1 according to the invention, for the two protectors. Conversely, without using statistical tests, the data show that the mass of the example of formulation 2 is significantly less stable than the mass of example 1 according to the invention, for the two protectors, since the level of probe tack decreases over time.

TABLE I

| | | | |
|---|---|---|---|
| Probe tack (10 mm) | Example 1 according | Protector Silphan | 104 |
| in kPa at T0 (date of | to the invention | Protector R1010 | 67 |
| manufacture) | Example 2 according | Protector Silphan | 139 |
| | to the invention | Protector R1010 | 98 |
| Probe tack (10 mm) | Example 1 according | Protector Silphan | 94 |
| in kPa after 18 | to the invention | Protector R1010 | 53 |
| months of aging at | Example 2 according | Protector Silphan | 121 |
| 25° C. | to the invention | Protector R1010 | 86 |
| Probe tack (10 mm) | Example 1 according | Protector Silphan | 102 |
| in kPa after 24 | to the invention | Protector R1010 | 70 |
| months of aging at | Example 2 according | Protector Silphan | 108 |
| 25° C. | to the invention | Protector R1010 | 67 |

1-10. (canceled)

11. A wound dressing which comprises a hydrocolloid adhesive mass comprising the following constituents, the percentages by weight of each constituent being indicated relative to the total weight of the hydrocolloid adhesive mass:
at least 10% and at most 30% of a hydrophobic elastomer matrix comprising poly(styrene-olefin-styrene) block copolymers,
at least 2% and at most 50% of particles of hydrocolloid(s) dispersed in said elastomer matrix,
at least 10% and at most 40% of at least one tackifying hydrocarbon resin,
characterized in that the hydrocolloid adhesive mass has a deformation ratio γ comprised between at least 0.10 and at most 0.40, as measured by subjecting a cylindrical sample having a diameter of 10 mm, of said hydrocolloid adhesive mass having a thickness of 1.0 mm, at a temperature of 37° C. in an atmosphere having a relative humidity of 60%, to a stress of 5000 Pa in order to cause a creep with which is associated a deformation under stress γ_{C} measured after 30 minutes of stress, followed by removing the stress in order to enable recovery characterized by a residual deformation γ_{R} after 30 minutes with no stress, the non-dimensional deformation ratio γ being given by:
$$γ = \frac{{γ}_{C} - {γ}_{R}}{{γ}_{C}}$$

12. The dressing of claim 11, in which the dressing and the adhesive mass which it contains are in a three-dimensional form having thinned edges, such that the height profile of the dressing going from the geometric center of the dressing to the periphery of the dressing consists of three sections:
a central part comprising the geometric center of the dressing, in which the height E of the profile (maximum thickness of the dressing over its central part) is substantially constant;
a peripheral part having a substantially constant thickness e;
a transition zone in which the curvature of connection linking the peripheral and central parts can be represented by a parabolic function of general formula (1):
y=a·x²+b·x+c, with a<0
in which x represents the horizontal distance on the profile, and y represent the height (thickness of the dressing) in the transition zone,
it being specified that:
0.0010 mm⁻¹≦−a≦0.0036 mm⁻¹, and
0.060≦b≦0.130.

13. The dressing of claim 12, in which the height E in the central part of the dressing of substantially constant thickness is at least 0.80 mm and at most 1.2 mm, and the height e in the peripheral part of the dressing of substantially constant thickness is at least 0.26 mm and at most 0.40 mm.

14. The dressing of claim 12, in particular of oval or rectangular type, comprising distinct longitudinal and transverse axes passing through the geometric center of the dressing, in which:
on the longitudinal axis, 0.0010 mm⁻¹≦−a≦0.0016 mm⁻¹, and 0.060≦b≦0.0920, and
on the transverse axis, 0.0020 mm⁻¹≦−a≦0.0036 mm⁻¹, and 0.080≦b≦0.130.

15. The dressing of claim 13, in particular of oval or rectangular type, comprising distinct longitudinal and transverse axes passing through the geometric center of the dressing, in which:
on the longitudinal axis, 0.0010 mm⁻¹≦−a≦0.0016 mm⁻¹, and 0.060≦b≦0.0920, and
on the transverse axis, 0.0020 mm⁻¹≦−a≦0.0036 mm⁻¹, and 0.080≦b≦0.130.

16. The dressing of claim 11, in which said poly(styrene-olefin-styrene) block copolymers comprise poly(styrene-isoprene-styrene) triblock copolymers and/or poly(styrene-isoprene) diblock copolymers.

17. The dressing of claim 15, in which said poly(styrene-olefin-styrene) block copolymers comprise poly(styrene-isoprene-styrene) triblock copolymers and/or poly(styrene-isoprene) diblock copolymers.

18. The dressing of claim 11, in which said particles of hydrocolloid(s) comprise at least one entity chosen from the group made up of: pectin, alginates, natural plant gums such as karaya gum, cellulose derivatives such as carboxymethylcelluloses and their alkali metal, in particular sodium or calcium, salts, and also synthetic polymers based on acrylic acid salts.

19. The dressing of claim 17, in which said particles of hydrocolloid(s) comprise at least one entity chosen from the group made up of: pectin, alginates, natural plant gums such as karaya gum, cellulose derivatives such as carboxymethyl [symbol] celluloses and their alkali metal, in particular sodium or calcium, salts, and also synthetic polymers based on acrylic acid salts.

20. The dressing of claim 11, also comprising one or more of the following agents: antioxidant(s), plasticizer(s) and surfactant(s).

21. The dressing of claim 19, also comprising one or more of the following agents: antioxidant(s), plasticizer(s) and surfactant(s).

22. The dressing of claim 11, in which the dressing comprises a backing layer on the face of the hydrocolloid adhesive mass not intended to be in contact with the patient's skin.

23. The dressing of claim 21, in which the dressing comprises a backing layer on the face of the hydrocolloid adhesive mass not intended to be in contact with the patient's skin.

24. The dressing of claim 11, in which the dressing comprises a protective film on the face of the hydrocolloid adhesive mass intended to be in contact with the patient's skin.

25. The dressing of claim 23, in which the dressing comprises a protective film on the face of the hydrocolloid adhesive mass intended to be in contact with the patient's skin.

26. A method for treating wounds, such as exudative wounds, burns, superficial or deep dermo-epidermal lesions, which may be chronic or acute, in particular in a method for treating blisters, by applying the wound dressing of claim 11.

27. A method for treating wounds, such as exudative wounds, burns, superficial or deep dermo-epidermal lesions, which may be chronic or acute, in particular in a method for treating blisters, by applying the wound dressing of claim 25.
